(19) United States
(12) Patent Application Publication
(10) Pub. No.: US 2018/0271068 A1
(43) Pub. Date: Sep. 27, 2018

(54) Genetically-Edited Swine

(71) Applicant: The University Court of the University of Edinburgh, Edinburgh (GB)

(72) Inventors: Christopher Bruce Alexander Whitelaw, Biggar South Lanarkshire (GB); Simon Geoffrey Lillico, Midlothian (GB)

(21) Appl. No.: 15/762,363

(22) PCT Filed: Sep. 29, 2016
(86) PCT No.: PCT/GB2016/053025

§ 371 Date: Mar. 22, 2018

(30) Foreign Application Priority Data
Sep. 29, 2015 (GB) 1517227.3

(51) Int. Cl. A01K 67/027; C07K 14/47

(52) U.S. Cl. CPC A01K 67/0275; C07K 14/4702; A01K 2217/07; A01K 2267/02; A01K 2227/108

(57) ABSTRACT

The present invention relates to genetically-edited swine comprising an introgressed heterologous nucleic acid sequence in the RELA gene. In particular it relates to genetically-edited swine comprising an introgressed warthog allele in the RALA gene of domestic pigs. The invention also related to methods of producing such swine, and cells derived from swine having such introgressed sequences.

The present invention relates to genetically-edited swine comprising an introgressed heterologous nucleic acid sequence in the RELA gene.

### BACKGROUND OF THE INVENTION

Classical animal breeding utilises sequence variation across the entire genome. Offspring resulting from mating of two animals have a genotype which is a mix of both parents plus de novo mutations. In agriculture beneficial genotypes and their encoded traits are captured conferring genetic improvement. This process is time consuming, requires multiple crosses, and relies on the presence of the desired genetic variation in the breeding population; variation that is eliminated during the breeding process cannot be exploited.

The development of targeted genome editing¹, pioneered by the zinc finger nuclease (ZFN) approach², now enables variation not present within a given population to be used. This approach relies on engineered nucleases to direct a double-strand break (DSB) to a specific target sequence. When used in combination with an investigator-provided DNA template, specified changes can be introduced into the chromosome in a homology-based process during repair of the DSB. In this way the original target sequence can be exchanged for a new sequence³, enabling single allele introgression into the target animal population in one generation 4.

Nevertheless, despite the promises of targeted genome editing, there remains significant challenges to performing allele introgression into target animals. In particular, allele introgression in domestic pigs (Sus scrofa) has not yet been successfully achieved.

While single point mutations have been found to confer key phenotypic differences during domestication of wild species⁵, in many cases, multiple point mutations in the same locus are thought to be responsible. A representative, agronomically important example is variation in RELA⁶. The domestic pig is highly susceptible to infection by African Swine Fever Virus, in contrast to present-day pig species found in Africa. We have earlier identified three amino acid differences between warthog (Phacochoerus africanus) and domestic pig RELA⁶.

WO2014/041327 describes genome editing of pigs by creating indels via NHEJ after DNA cleavage by ZFNs and TALENs, but does not describe allele introgression.

The present inventors have achieved success in carrying out allele introgression in the RELA gene of pigs. This invention thus overcomes considerable uncertainty to complete this landmark achievement.

### STATEMENTS OF THE INVENTION

The inventors have described for the first time the introgression of a heterologous nucleic acid into the swine genome; in particular, they have introgressed a complete haplotype of the warthog RELA allele into the domestic pig RELA gene. The introgression of such a haplotype into the pig genome is a remarkable achievement, and the resultant pigs are of considerable commercial interest.

According to a first aspect of the present invention there is provided a genetically-edited swine comprising an introgressed heterologous nucleic acid sequence in the RELA gene.

It is highly preferred that the swine is a pig, and more preferably a domestic pig.

The RELA protein is a predominant component of the NFkappaB heterodimeric transcription factor. As such, genetic editing which alters the levels or activity of RELA will directly affect NFkappaB dependent cell activities, in particular transcription from NFkappaB induced genes. NFkappaB is a key effector of animal responses to various stresses, including infection. Genetically-edited animals with altered RELA expression or activity will therefore react differently to their non-edited counterparts in response to biological stresses or insults, such as infection, chronic and/or autoimmune diseases.

It is highly preferred that the introgressed heterologous nucleic acid sequence comprises a heterologous RELA allele. Suitably, the introgressed heterologous allele comprises a trans-species heterologous RELA allele. Optionally, the introgressed heterologous allele comprises a trans-genus heterologous RELA allele.

Accordingly, in particularly preferred embodiments of the invention the introgressed heterologous allele converts a wild-type RELA allele to a heterologous RELA allele, more preferably a trans-species heterologous RELA allele or a trans-genus heterologous RELA allele, and suitably the warthog RELA allele.

It is a particularly preferred feature of the present invention that an allele of the RELA gene (which can include regulatory and non-coding sequences) present in an animal (e.g. a domestic pig) can be ‘re-written’ via introgression such that a different allele is present—in many cases this may involve changes to only small number of bases. This can be done in a completely ‘clean’ manner, i.e. no footprint or other trace of the editing event is left behind, and the only changes made to the genome are those required for the desired allele conversion. In this way, for example, a RELA allele that is naturally found in one species, can be introduced into a population (e.g. species) in which the allele is not present. This approach is very different from conventional transgenesis in which genes (and often selectable markers) are inserted, moved or disrupted, but wherein some form of footprint, in the sense of genomic disruption, results.

The heterologous allele of the present invention is not formed by a deletion, inversion, or other such random or poorly-controlled edits which are the typical result of non-homologous end joining (NHEJ). The introgressed heterologous allele is typically introgressed by allele conversion via homology directed repair (HDR) of a site-specific nuclease (SSN) induced double stranded break (DSB) in the genomic DNA at or near the locus of said allele based upon a template nucleic acid (typically DNA) sequence comprising the sequence of the heterologous allele.

Preferably the genetically-edited swine comprises an introgressed RELA allele that differs from the wild-type RELA allele sequence by changes in two or more bases. The swine thus preferably comprises a introgressed RELA haplotype. The haplotype is preferably introgressed via a single editing event. The haplotype can contain 2, 3, 4, 5, 6, 7, 8, 9, 10, 15, 20, 30 or more base changes compared with the wild-type RELA sequence.

In some embodiments of the present invention the genetically-edited swine comprises an introgressed nucleic acid in the RELA gene which is 50 or more bases in length, suitably 100 or more, 150 or more, 200 or more, 250 or more, 500 or more, or 1000 or more, or 1500 or more bases in length. Where the invention relates to an introgressed haplotype, the distance spanning the 2 or more bases which are altered can be 50 or more bases, optionally 100 or more, 150 or more, 200 or more, 250 or more, 500 or more, or 1000 or more bases.

Preferably all cells of the genetically-edited swine contain the introgressed heterologous nucleic acid sequence. This can be achieved, for example, by modifying the single-cell zygote and raising a swine from the zygote.

The introgressed RELA allele preferably changes the sequence of the RELA protein encoded by the RELA gene. It is generally preferred that the introgressed RELA allele results in an alteration to the coding region (exons) of the RELA gene, i.e. corresponding to the cDNA sequence set out in SEQ ID NO 15. The introgressed RELA allele thus preferably results in a change of one or more amino acids relative to the wild-type domestic pig RELA amino acid sequence shown in SEQ ID NO 16.

It is highly preferred that the introgressed RELA allele changes sequences in the region of the RELA gene which encodes the transactivation domain of RELA. In pigs, such domains extend from amino acid 431 to 553 of the wild-type RELA protein sequence (unless otherwise stated, nucleic acid and amino acid numbering is with reference to the wild-type Sus scrofa RELA cDNA or protein sequences). Transactivation domain 2 extends from amino acid 431 to 521 and transactivation domain 1 extends from amino acid no 522 to the C-terminus of the protein at amino acid 553. More preferably, the introgressed RELA allele changes the region of the RELA gene which encodes amino acids 448 to 531 of RELA.

Suitably the genetically-edited swine is a pig that comprises an introgressed trans-species allele of the RELA gene. In other words, the pig comprises an introgressed heterologous nucleic acid sequence which converts the domestic pig RELA sequence to the sequence of a trans-species allele. More preferably the pig comprises an introgressed trans-species allele of the RELA gene from a swine species outside of the genus Sus. Most preferably the pig comprises an introgressed trans-species allele of the warthog RELA gene, and in particular an allele of the sequence encoding the transactivation domain of RELA.

In some embodiments of the invention the introgressed heterologous nucleic acid sequence is a sequence which is not present within the breed, sub-species, and preferably the species, of the genetically-edited swine (e.g. it is non-native to the relevant species). For example, the introgressed heterologous nucleic acid sequence suitably comprises a heterologous RELA allele that is not present in domestic pigs.

The swine can be heterozygous (mono-allelic) or homozygous (bi-allelic) for the introgressed RELA allele. Preferably the swine is homozygous (bi-allelic) for the introgressed RELA allele.

In a preferred embodiment the modification causes a change in the amino acid located at one or more of the following amino acids of RELA:
T448;
S485; and
S531.

Suitably the amino acids at two or more of these sites are altered. However, it is highly preferred that the amino acids at all three of the sites are altered.

The modifications may suitably result in the following changes in the amino acids of RELA: T448A, S485P, and/or S531P. These alterations correspond to polymorphisms that have been observed between domestic pigs and warthogs. These polymorphisms correlate with tolerance to ASFV infection in warthogs.

In some embodiments the modifications may suitably result in one of the following changes in the amino acids of RELA: T448A; S485P; S531P; T448A and S485P; S485P and S531P; T448A and S531P; T448A, S485P and S531P.

In a highly preferred embodiment the swine is a pig that comprises an introgressed heterologous nucleic acid sequence which results in the following amino acid changes to the domestic pig RELA protein: T448A, S485P and S531P. These three changes are the amino acid differences between the wild-type RELA protein sequence in domestic pigs and the wild-type RELA protein sequence in warthogs. It is preferred that no amino acid changes other than T448A, S485P and S531P are caused by the introgressed heterologous nucleic acid.

Where a genetic editing event means that these three amino acid changes are made (and no others) to a domestic pig, then it can be said that a perfect trans-genus allele conversion has occurred, i.e. perfectly converting the domestic pig RELA allele to the corresponding warthog allele in the absence of any unintended changes. Warthogs contain several other polymorphisms at the nucleic acid level but they do not affect the expressed amino acid sequence and thus in the present case can be ignored.

In a preferred embodiment, the present invention thus provides a genetically-edited pig wherein the RELA gene has been edited such that it comprises the sequence as set out below (the amino acids at sites 448, 485 and 531 are shown in bold):

LLQLQFDADEDLGALLGNNTDPTVFTDLASVDNSEFQQLLNQGVPMPPHT
AEPMLMEYPEAITRLVTGSQRPPDPAPTPLGASGLTNGLLPDGEDFSSIA
DM (i.e. including changes T449A, S485P and S531P-
SEQ ID NO 17)

Accordingly, the present invention thus provides a domestic pig which has been genetically-edited such that at least a portion of the autologous RELA sequence that includes the sequences encoding S531, T449 and S485 has been replaced by introgression (suitably via HDR of a DSB induced by a suitably targeted SSN) of a sequence which encodes the corresponding warthog (Phacochoerus sp.) RELA protein sequence. The introgressed nucleic acid sequence can be identical to the warthog sequence or can be an equivalent artificial sequence comprising one or more synonymous base changes.

In a particularly preferred embodiment of the present invention, the genetically-edited swine is a pig (preferably a domestic pig) which has improved tolerance to ASFV infection resulting from the introgressed heterologous nucleic acid.

An animal can be said to be more tolerant to infection when the morality rate, morbidity rate, the proportion of animals showing significant morbidity (e.g. weight loss or decreased growth rate), the level of morbidity or the duration of morbidity is reduced. In the case of ASFV in domesticated pigs, the morbidity rate approaches 100% in naive herds. The mortality rate depends on the virulence of the isolate, and can range from 0% to 100%. Highly virulent isolates can cause almost 100% mortality in pigs of all ages. Less virulent isolates are more likely to be fatal in pigs with a concurrent disease, pregnant animals and young animals. In sub-acute disease, the mortality rate may be as high as 70-80% in young pigs, but less than 20% in older animals. Any statistically significant reduction (e.g. 95% confidence, or 99% confidence using an appropriate test) in the mortality or morbidity between a population of genetically-edited pigs and a population of equivalent non-edited pigs when exposed to ASFV of the same virulence level (ideally the same isolate) demonstrates improved tolerance.

According to a second aspect of the invention there is provided a cell nucleus, germ cell, stem cell, gamete, blastocyst, embryo, foetus and/or donor cell of a swine comprising an introgressed heterologous nucleic acid sequence in the RELA gene.

Preferred features of the second aspect of the present invention correspond to those of the first aspect of the invention.

In a preferred embodiment the cell nucleus, germ cell, stem cell, gamete, blastocyst, embryo, foetus and/or donor cell is from a domestic pig, and comprises an introgressed trans-species RELA allele, e.g. the warthog RELA allele.

Suitably the cell nucleus, germ cell, stem cell, gamete, blastocyst, embryo, foetus and/or donor cell is derived from a swine as set out above. Alternatively, it can be created de novo using the methods described herein.

According to a third aspect the invention provides a method of producing a genetically-edited swine having an introgressed heterologous nucleic acid sequence in the RELA gene, the method comprising the steps of:
providing a swine zygote;
introducing into said zygote a site-specific nuclease, the nuclease being adapted to target a desired genomic sequence in the RELA gene to be edited, and to introduce a double stranded break;
introducing a template nucleic acid comprising the heterologous nucleic acid adapted to introgress the heterologous nucleic acid sequence into the RELA gene, the heterologous sequence being flanked by sequences homologous to genomic RELA sequences;
incubating said zygote under suitable conditions to permit cutting of the genome by the site-specific nuclease and introgression of the heterologous nucleic acid sequence into the RELA gene by homology directed repair; and
generating an animal from said zygote.

Preferably the swine is a pig, and more preferably a domestic pig.

Preferably the site-specific nuclease comprises a zinc finger nuclease (ZFN), a Transcription Activator-Like Effector Nuclease (TALEN), an RNA-guided CRISPR/Cas nuclease (CRISPR), or a meganuclease. More preferably, the site-specific nuclease comprises a pair of cooperating ZFNs, TALENs or RNA-guided CRISPR ‘nickases’ (e.g. having a modified Cas9 nuclease capable of cutting only one DNA strand), adapted such that DNA cutting only occurs when both members of the pair are present and form a heterodimer, which is able to cut both strands of the DNA molecule. The use of a pair of cooperating ZFNs, TALENs or RNA-guided CRISPRs results in a reduction of possible off-target cutting events. In some preferred embodiments the site-specific nuclease comprises a pair of ZFNs.

Preferably the site-specific nuclease is adapted to target and cut within the region of the RELA gene encoding the transactivation domain of RELA (i.e. amino acid 431 to 553 of the wild-type RELA protein sequence) or slightly upstream or downstream thereof, e.g. within 500, 300, 200, 100, 50 or 20 bases upstream or downstream thereof. More preferably the site-specific nuclease is adapted to target and cut within region of the RELA gene which encodes amino acids 448 to 531 of RELA, or slightly upstream thereof, e.g. within 500, 300, 200, 100, 50, 20 or 10 bases upstream thereof.

Preferably the site-specific nuclease is adapted to target and cut within exon 9 of the RELA gene.

In a preferred embodiment the site-specific nuclease is adapted to target and cut upstream of the region of the RELA encoding amino acid T448, e.g. within 500, 300, 200, 100, 50 or 20 bases upstream thereof.

In a particular preferred embodiment, the site-specific nuclease is adapted target a suitable sequence to cut at a site lying between bases 1200 and 1341 (with reference to SEQ ID NO 15), more preferably at a site lying between bases 1250 and 1340, yet more preferably at a site lying between bases 1300 and 1340, and yet more preferably at a site lying between bases 1320 and 1340. In one embodiment the cut site lies between bases 1332 and 1333.

In one specific embodiment of the present invention the target site of one of a pair of cooperating SSNs is GATACTGATGAGGAC (SEQ ID NO 18) and the target site of the other of the pair of SSNs is CTCCGGGACGACGTC (SEQ ID NO 19). Other target sequences could be used, and the skilled person is readily able to determine suitable target sites optimised for different SSNs.

It should be noted that the site-specific nuclease can be introduced to a cell in any suitable form. For example, the nuclease can be provided directly into the zygote as a functional protein. Alternatively, the nuclease can be provided into the zygote in the form of a precursor or template from which the active nuclease is produced by the zygote. In a preferred embodiment an mRNA encoding the nuclease is introduced into the zygote, e.g. by injection. The mRNA is then translated by the cell to form the functioning protein. Using mRNA in this way allows rapid but transient expression of the nuclease within the cell, which is ideal for the purposes of genetic editing.

The term ‘zygote’ can be used in a strict sense to refer to the single cell formed by the fusion of gametes. However, it can also be used more broadly to refer to the cell bundle resulting from the first few divisions of the true zygote (this is more properly known as the morula).

It is preferred that the present method is at least initiated, and preferably completed, in the zygote at the single cell stage.

The genetically-edited zygote can be grown to become an embryo and eventually an adult animal. If the editing event occurs in the single-cell zygote then all cells of this animal will comprise the modified RELA gene as all cells of the animal are derived from a single genetically-edited cell. If the editing event occurs after one or more cell divisions then the resultant animal will likely be a mosaic for the editing event, in that it will have some cells derived from the edited cell and some cells derived from unedited cells.

The method can be performed on a plurality of zygotes and the method may involve selecting zygotes in which the desired genetic modification has been achieved.

Preferably the template nucleic acid comprises a region including the heterologous nucleic acid sequence flanked on each side by homologous sequences. The template construct can comprise a heterologous nucleic acid sequence that is, for example, 50 or more bases in length, suitably 100 or more, 150 or more, 200 or more, 250 or more, 500 or more, or 1000 or more, or 1500 or more bases in length. The flanking homologous sequences can be, for example, 50 or more bases in length, suitably 100 or more, 150 or more, 200 or more, 250 or more, 500 or more, or 1000 or more bases in length.

Preferably the template nucleic acid comprises a region including the warthog RELA haplotype flanked on each side by homologous regions. It is important to note that a region including the warthog RELA haplotype would typically be largely homologous to the wild-type target sequence, except for changes at the necessary bases to achieve the desired edit(s).

The homologous region can be, for example, from 200 to 1000 bases in length, suitably from 500 to 900 bases in length.

In one particular embodiment the template nucleic acid comprises a region including the warthog RELA of 251 or more nucleotides in length, which comprises a nucleic acid sequence encoding the protein sequence ADEDLGALLGNNTDPTVFTDLASVDNSEFQQLLNQGVPMPPHTAEPMLMEYPEAITRLVTG SQRPPDPAPTPLGASGLTNGLLP (SEQ ID NO 23) (the amino acids changes T448A, S485P and S531P are shown in bold) flanked by homologous regions. The homologous regions can be 200 bases or longer, suitably 400 bases or longer, more preferably 600 bases or longer.

Preferably the template nucleic acid is double stranded.

Preferably the template nucleic acid is provided in a plasmid. Provision of the template in the form of a plasmid has been found to result in improved efficacy and/or efficiency of introgression.

In one particular embodiment the template is plasmid comprising a 251 bp region containing the warthog RELA haplotype (i.e. encoding SEQ ID NO 23) flanked by regions (homology arms) of 626 bp and 799 bp. The 251 bp region contains 5 base changes to convert the domestic pig sequence to the warthog haplotype.

It is highly preferred that the template nucleic acid comprises one or more (preferably two or more, yet more preferably three or more) base changes compared with the corresponding genomic nucleic acid sequence at the target site for the SSN. Provision of such changes means that following HDR the target site for the SSN will be destroyed (or at least rendered sub-optimal), thus preventing or reducing re-cutting once a successful introgression has occurred.

In a preferred embodiment of the present invention, where a target site for the SSN is GATACTGATGAGGAC (SEQ ID NO 18) the template nucleic acid comprises the sequence GATgCaGAcGAGGAC (SEQ ID NO 20) to replace the genomic sequence and prevent re-cutting by the SSN. For clarity, the corresponding sequences pre- and post-introgression are shown below (SSN target site is underlined, the cut site is in bold, and the changes are shown in lower case):

Genomic
(SEQ ID NO 21)
TGCAGTTTGATACTGATGAGGAC
Post-introgression
(SEQ ID NO 22)
TGCAGTTTGATgCaGAcGAGGAC

Accordingly, in a particularly preferred embodiment of the present invention, there is provided a method of producing a genetically-edited domestic pig having an introgressed warthog RELA haplotype, the method comprising the steps of:
providing a domestic pig zygote;
introducing into said zygote a pair of cooperating site-specific nucleases (suitably ZFNs), the nucleases being adapted to target the RELA gene in the region of (preferably upstream, and suitably within 20 bp) of the sequence encoding T448A and introduce a double stranded break;
introducing a template nucleic acid (preferably a double-stranded DNA template, e.g. a plasmid) comprising a heterologous nucleic acid comprising a sequence encoding a corresponding warthog RELA haplotype flanked by sequences homologous to the genomic RELA sequence of the pig;
incubating said zygote under suitable conditions to permit cutting of the genome by the site-specific nuclease and introgression of the heterologous nucleic acid by homology-directed repair; and
generating a pig from said zygote.

Embodiments of the present invention will now be described, by way of non-limiting example, with reference to the accompanying drawings.

### BRIEF DESCRIPTION OF THE FIGURES

FIG. 1—Design strategy for creation of a DSB and subsequent HDR of the domestic pig RELA. (a) Depiction of the RELA gene with exons as solid bars. An expanded view of a region of the final exon highlights the 3 amino acid differences between domestic pig (red) and warthog (green). Scissors indicate the intended site of the DSB. (b) DNA sequence, with solid bars representing ZFN binding sites on the domestic pig sequence. Lower panel indicates sequence changes that would concurrently change the domestic pig threonine to the warthog alanine whilst at the same time prevent re-cutting by the ZFN. (c) Design of HDR template, with 626 bp and 799 bp homology arms flanking a 251 bp region, including 5 base changes to convert the domestic pig sequence to the warthog haplotype.

FIG. 2—Sequence analysis of live born piglets. The sequence of both the domestic pig and warthog encoding the three observed amino acid differences is shown above, with sequence traces from individual animals below. Inset pictures of chromosomes indicate the allelic makeup at each position in each animal (domestic pig allele—red; warthog allele—green).

### SPECIFIC DESCRIPTION OF EMBODIMENTS OF THE INVENTION

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. The term “swine”, or variants thereof, as used herein refers to any of the animals in the Suidae family of even-toed ungulates including animals in the genus Sus and other related species, including the peccary, the babirusa, and the warthog.

The term “pig” or variants thereof as used herein refers to any of the animals in the genus Sus. It includes the domestic pig (Sus scrofa domesticus or Sus domesticus) and its ancestor, the common Eurasian wild boar (Sus scrofa). For the present purposes the domestic pig is considered to be a sub-species of the species Sus scrofa. It does not include the peccary, the babirusa, and the warthog.

The term “domestic pig”, or variants thereof, as used herein refers to an animal of the sub-species Sus scrofa domesticus.

The term “RELA gene”, or variants thereof, as used herein refers to the RELA (V-Rel Avian Reticuloendotheliosis Viral Oncogene Homolog A gene, also known as the p65 gene, NCBI Gene ID: 100135665) gene, and includes both coding and non-coding regions, and also associated regulators promoter and enhancer regions. In preferred embodiments of the invention introgression modifies the sequence within the RELA gene ORF, and more preferably within at least one exon.

The term “site-specific nuclease”, or variants thereof, as used herein refers to engineered nucleases which can be configured to cut DNA at a desired location. Such site-specific nucleases are also known as engineered nucleases, targetable nucleases, genome editing nucleases, molecular scissors, and suchlike. Examples of site-specific nucleases include zinc finger nucleases (ZFNs), Transcription Activator-Like Effector Nucleases (TALENs), the CRISPR/Cas system (CRISPR), and meganucleases, such as hybrid meganucleases.

The term “heterologous allele”, or variants thereof, as used herein refers to an allele which is not present in the relevant animal. The heterologous allele can be naturally occurring in another species or genus, or it can be non-natural in any species (i.e. entirely artificial). Preferably the allele is naturally occurring in another species.

The term “trans-species heterologous allele”, or variants thereof, as used herein refers to an allele which does not naturally occur in the species of the relevant animal, but which occurs naturally in another species. The heterologous allele can be naturally occurring in another species, which species may be from the same or a different genus. Thus a trans-species allele is still a ‘natural allele’ in the sense that it is not artificial and is found in nature, but it is introgressed to a new species to form a new animal with desired properties.

The term “trans-genus heterologous allele”, or variants thereof, as used herein refers to an allele which does not naturally occur in the genus of the relevant animal, but which occurs naturally in another genus. The set “trans-genus heterologous alleles” is thus a subset of “trans-species heterologous alleles”, i.e. wherein a trans-genus heterologous allele comes from outside of the relevant animal's genus, and not merely from outside of the relevant animal's species. For example, the RELA allele from warthogs is a trans-genus heterologous allele for animals in the genus Sus, and in particular to domestic pigs.

The term “haplotype”, or variants thereof, as used herein refers to a linked set of DNA sequence variations (typically single-nucleotide polymorphisms (SNPs)) at a specific locus on a single chromatid of a chromosome pair. In the present invention a haplotype is typically a plurality of SNPs differing between one species or genus and other, which contribute to or define a heterologous allele as between the one species or genus and the other. For example, in the case of the RELA allele in the present example there are 3 amino acid changes as between domestic pigs and warthog; these changes represent the haplotype of the heterologous RELA allele as between domestic pigs and warthogs.

The term “introgression”, or variants thereof, as used herein refers to the introduction of a heterologous nucleic acid sequence, especially a gene or allele, from a given source into an animal, typically by rewriting or converting an existing genomic sequence. Re-writing or converting in the present invention is achieved by HDR. The source of the heterologous nucleic acid sequence can be an animal from another species or genus, or it can be an artificial sequence.

The term “allele introgression”, or variants thereof, as used herein refers to a genetic edit which introduces an allele to the genome of an animal. The allele introgression can be an “allele conversion” or “allele replacement”, or it may, for example, introduce a new gene in its entirety. In preferred embodiments of the present invention the allele introgression is an allele conversion or allele replacement.

The term “allele conversion” or “allele replacement”, or variants thereof, as used herein refers to an introgression which replaces a normal, usually ‘wild-type’, allele with a heterologous allele. Conversion or replacement of a wild-type allele to a heterologous allele can in some cases involve alteration of the wild-type genomic DNA sequence to exactly match the DNA sequence of the heterologous allele from another animal. However, in other cases conversion or replacement may only require modification to the wild-type genomic DNA sequence such that the encoded protein matches the protein encoded by the heterologous allele (i.e. synonymous substitutions need not be made to the wild-type genomic sequence). The type of alteration required will depend on the manner in which the allele exerts its phenotype, e.g. via the activity of encoded protein versus regulation of transcription; in the former the encoded protein sequence is of primary importance, whereas in the latter the DNA sequence is of primary importance.

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

Pioneering work by the Jasin laboratory demonstrated that a single DSB in mammalian cells can lead to the transfer of a panel of single nucleotide polymorphisms (SNPs) forming an uninterrupted, short (<200 bp) haplotype to the chromosome from an extrachromosomal repair template⁷.

The present inventors sought to use genome editing to introduce the entire warthog RELA haplotype (which spans 251 base pairs, bearing five SNPs resulting in 3 amino acid changes) via a single nuclease-induced DSB. To the best of our knowledge, such editing-driven haplotype introgression had not been previously reported in live born animals of any mammalian species. We now demonstrate that this can be achieved efficiently in domestic pig by direct injection into the zygote cytoplasm. Strikingly, we observe single-step bi-allelic haplotype transfer by genotyping of piglets. Detailed materials and methods are provided in a separate section below, and the outline methodology, results and conclusions will now be discussed.

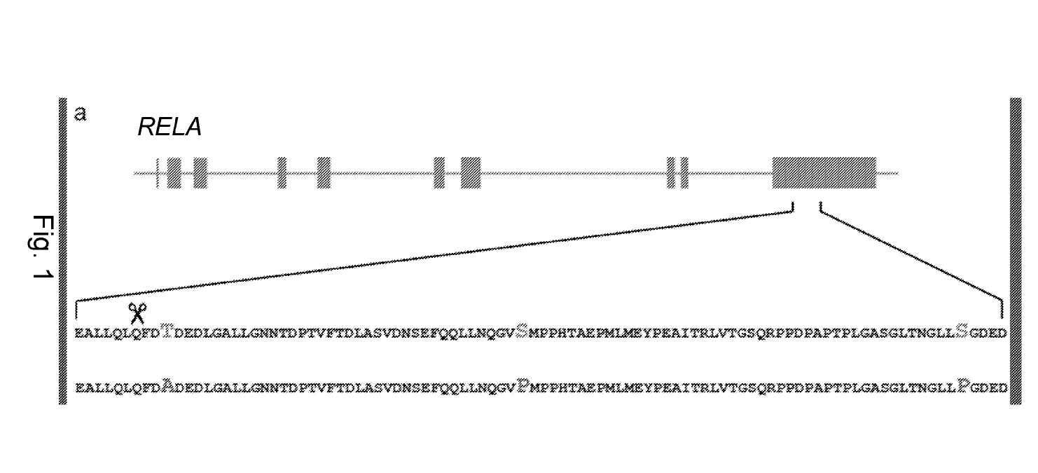

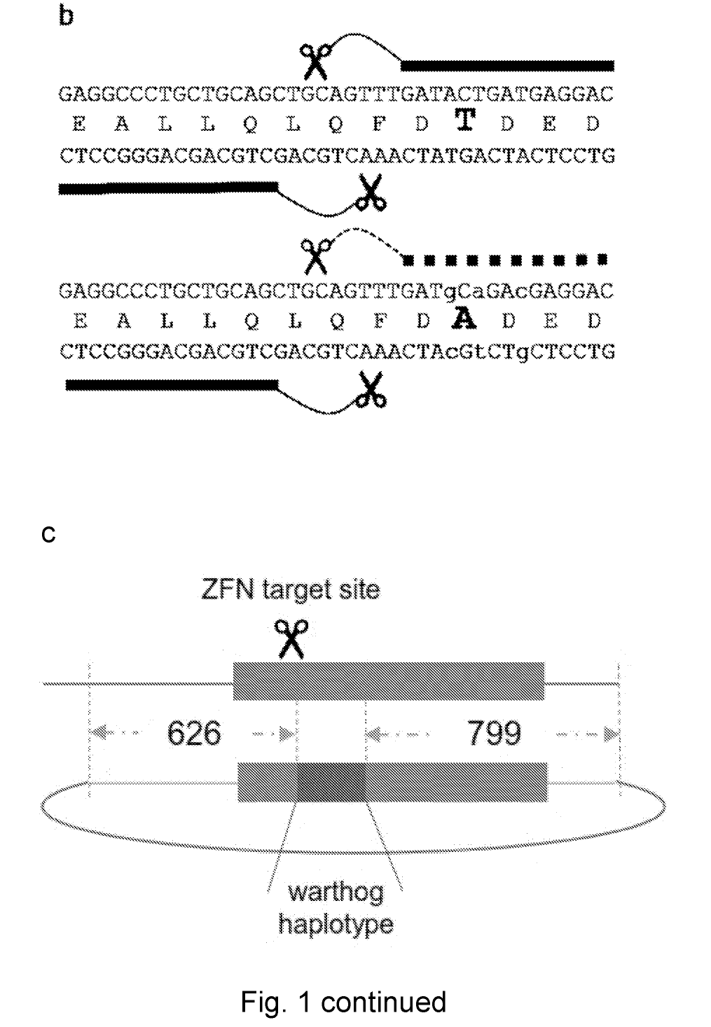

ZFNs can be engineered to induce a DSB at any genomic position. In this specific case, nuclease design considerations were informed by the need to transfer an entire 251 bp haplotype bearing multiple SNPs. The inventors conceived a strategy (FIG. 1a) in which a ZFN is engineered for a region immediately upstream of the haplotype-marked stretch, with the intention that single-sided invasion of the repair template by the upstream chromosome arm would then lead to a synthesis-dependent strand annealing-based transfer of the entire downstream haplotype to the endogenous locus.

A ZFN heterodimer (see Table 1 for details) was produced that binds to the region flanking 1330 to 1338 bp relative to the translational start site in the porcine RELA cDNA sequence (NM_001114281). We compared two formats of an expression construct for the ZFNs: two plasmids, each encoding a single ZFN monomer, and one plasmid that encodes both ZFN monomers spanned by a ribosome stuttering signal or a 2A peptide^{8, 9}. We transfected these plasmids into a transformed cell line (PK15) established from the domestic pig, and compared genome editing efficiencies via the Surveyor/Cel-1 endonuclease assay¹⁰. We observed comparable on-target editing driven by either expression construct configuration. The editing efficiency driven by the RELA-directed ZFNs nearly doubled that seen with ZFNs successfully used to obtain live pigs bearing a disruption of the GGTA gene¹¹, suggesting that these nucleases may be well-suited for in-embryo editing.

While the present inventors used ZFNs to create a DSB in the genome, such a cut can be achieved using the various other site-specific nucleases now well-known to the skilled person. For example, a suitable TALEN pair could readily be designed to target the same locus, and the CRISPR/Cas system could also be used by providing suitable guide RNA sequences to guide either wild-type or paired ‘nickase’ Cas nuclease(s). Accordingly, while ZFNs are disclosed in the present examples, and ZFNs exhibited highly desirable properties, the present invention is not be restricted to the use of ZFNs.

ZFN technology is described extensively in the literature and, inter alia, in the following patent documents: U.S. Pat. Nos. 6,479,626, 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, 6,479,626, 8,106,255, 20030232410, and 20090203140, all of which are incorporated by reference. ZFNs can be obtained commercially from Sigma-Aldrich (St. Louis, Mo., US) under the CompoZr® Zinc Finger Nuclease Technology branded products and services.

TALEN technology is described extensively in the literature and, inter alia, in the following patent documents: U.S. Pat. No. 8,420,782, U.S. Pat. No. 8,470,973, U.S. Pat. No. 8,440,431, U.S. Pat. No. 8,440,432, U.S. Pat. No. 8,450,471, U.S. Pat. No. 8,586,363, U.S. Pat. No. 8,697,853, EP2510096, U.S. Pat. No. 8,586,526, U.S. Pat. No. 8,623,618, EP2464750, US2011041195, US2011247089, US2013198878, WO2012/116274, WO2014110552, WO2014070887, WO2014022120, WO2013192316, and WO2010008562, all of which are incorporated by reference. TALENs can be obtained commercially from Thermo Fisher Scientific, Inc. (Waltham, Mass., US) under the GeneArt® TALs branded products and services (formerly marketed under the Life Technologies brand).

CRISPR/Cas technology is described extensively in the literature (e.g. Cong et al. ‘Multiplex Genome Engineering Using CRISPR/Cas Systems’, Science, 15 Feb. 2013: Vol. 339 no. 6121 pp. 819-823) and, inter alia, in the following patent documents: U.S. Pat. No. 8,697,359, US2010076057, WO2013/176772, U.S. Pat. No. 8,771,945, US2010076057, US2014186843, US2014179770, US2014179006, WO2014093712, WO2014093701, WO2014093635, WO2014093694, WO2014093655, WO2014093709, WO2013/188638, WO2013/142578, WO2013/141680, WO2013/188522, U.S. Pat. No. 8,546,553, WO2014/089290, and WO2014/093479, all of which are incorporated by reference. CRISPR/Cas systems can be obtained commercially from Sigma-Aldrich (St. Louis, Mo., US) under the CRISPR/Cas Nuclease RNA-guided Genome Editing suite of products and services, or from Thermo Fisher Scientific, Inc. (Waltham, Mass., US) under the GeneArt® CRISPR branded products and services.

A robust combination of efficient on-target marking and minimal toxicity to early embryogenesis can be achieved by the delivery of nuclease-encoding mRNA to the embryo¹². We transferred the ORFs encoding the RELA ZFNs into two distinct vectors for in vitro mRNA production (pVAX, which requires in vitro polyadenylation, and pGEM, which contains a polyA track of defined length). For both vectors, we generated constructs bearing single ZFNs, and constructs bearing both ZFNs on the same ORF separated by a self-cleaving 2A signal. Capped and polyadenylated mRNA was then in vitro transcribed from all constructs, and the on-target editing efficiency assessed by transient transfection into pig PK15 cells.

This was followed by Surveyor/Cel-1 and a deep-sequencing based assay to measure the percentage of edited chromatids. Robust editing efficiency, in all cases exceeding that driven by positive control ZFNs, was obtained with all four vector/ORF configurations. We have previously shown that delivery of engineered nucleases to the cytoplasm of livestock zygotes can result in the production of small insertions or deletions (indels) due to non-homologous end-joining-driven (NHEJ) break repair at the target site¹³⁻¹⁵. It was not clear whether this delivery method could also result in HDR if combined with a DNA template. We co-injected porcine zygotes with mRNA encoding the pair of ZFN and either a single stranded oligodeoxynucleotide (ssODN¹⁶) or plasmid DNA bearing the warthog SNPs. Injected zygotes were transferred to recipient gilts¹⁴.

To determine whether the nucleases drove targeted editing of pig RELA, ear notches were taken from piglets 2 days postpartum and genomic DNA was prepared. PCR spanning the target locus and sequencing of these products was used to identify either alleles bearing small indels (a result of NHEJ) or specific point mutations (a result of HDR events). Highly surprisingly, no indels were observed at the ZFN target site in any of the animals genotyped; this contrasts both with our ability to obtain edited animals bearing NHEJ-generated alleles, and our earlier experience with genome editing by indels in pig^{13, 14}.

The lack of indels at the nuclease target site was not due to a failure of the genome editing process itself, as four live piglets bore HDR-generated alleles of RELA (see Table 2 below).

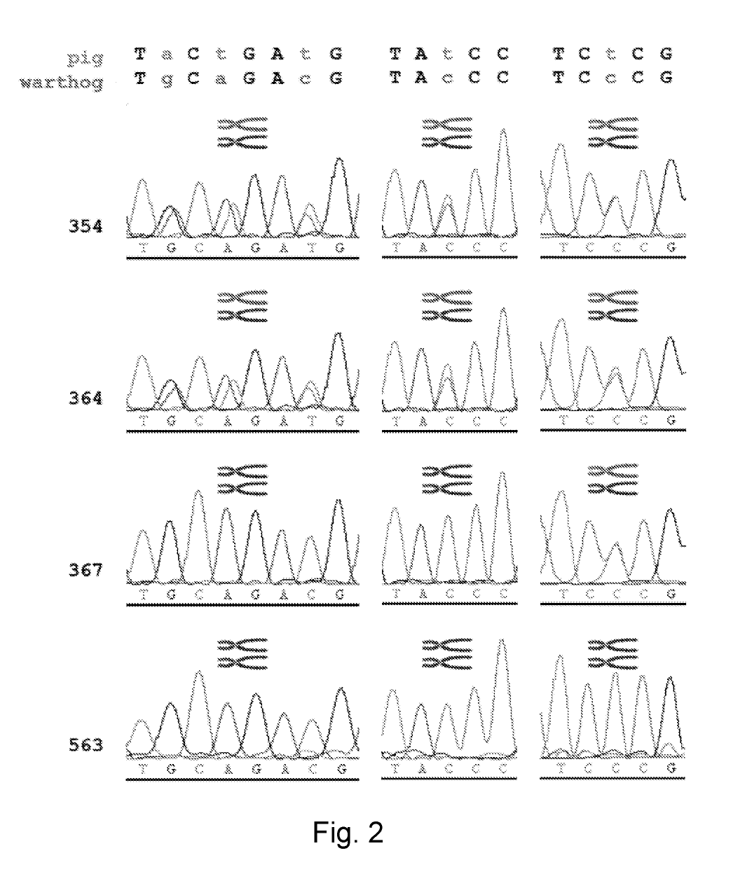

All four occurred in the cohort of 46 animals injected with ZFN-encoding mRNA and a plasmid repair template. In contrast, no HDR events were observed in the 39 live born pigs where ssODN was provided as the HDR template. Sanger sequencing of PCR products spanning the target locus of the HDR positive pigs showed that piglets 354 and 364 were heterozygous at each of the 5 base changes encoded by the plasmid template, thus representing full haplotype introgression (FIG. 2). Piglet 367 was homozygous for 4 base changes proximal to the ZFN target site, and heterozygous for the most distal modification. This finding demonstrated that continuity of gene conversion tracks from DSB-R in mammalian cells, and indicates that the two homologs were cleaved in the early embryo followed by distinct HDR-based resolution of the break. Remarkably, piglet 563 was homozygous for all 5 base changes.

Livestock breeding has enabled a continuous increase in animal productivity since animal domestication. The challenge ahead is to accelerate this improvement process to meet the demands imposed on agriculture through climate change, resource and land availability in conjunction with the increase in human population. Genome editing technology has the potential to revolutionize livestock breeding⁴, and targeted gene knockout in several livestock species has been attained using multiple distinct designed nuclease platforms, including ZFNs, TAL effector nucleases, and CRISPR/Cas9^{11, 13, 15, 17-19}.

The present inventors have significantly expanded the genome editors' toolbox to include the targeted transfer of an entire haplotype. Specifically, through homology dependent repair of a ZFN-induced break using a plasmid repair template we have introgressed an allele of the RELA gene between swine species, producing live piglets both heterozygous and homozygous for the desired haplotype.

### Materials and Methods

### ZFN Design and Validation.

ZFNs against the indicated position of the pig RELA gene were designed and assembled using an archive of pre-validated two-finger modules as described^{[2]}. The ORFs were cloned into expression vectors harbouring enhanced obligate heterodimer forms of Fokl^{[20]} optimized for delivery in DNA form and for production of in vitro transcribed mRNA (Vierstra et al., in press). ZFN target sequences and DNA recognition helices are described in Table 1. Pig PK15 cells were electroporated using ZFN-encoding DNA or mRNA as described, genomic DNA harvested 48 h following electroporation, and percentage of chromatids bearing indels was measured using Surveyor/Cel1 as described^{[10]} or deep sequencing on the Illumina platform.

TABLE 1a
ZFN target sequences (SEQ ID NOs are provided in brackets)

| ZFN Binding Sequence (underlined) | Targeting ZFN ID |
|---|---|
| AGAGGCCCTGCTGCAGCTGCAGTTTGATACTGATGAGGACC (1) | 48307 |
| TCTCCGGGACGACGTCGACGTCAAACTATGACTACTCCTGG (2) | 48304 |

TABLE 1b
ZFN DNA recognition helices

| ZFN ID | Finger 1 | Finger 2 | Finger 3 | Finger 4 | Finger 5 |
|---|---|---|---|---|---|
| 48307 | DRSDLSR (3) | RSDNLTR (4) | TSGNLTR (5) | LRQDLNK (6) | TSSNLSR (7) |
| 48304 | AMQTLRV (8) | DRSHLAR (9) | RSDNLSE (10) | KRCNLRC (11) | RSAVLSE (12) |

### Design and Construction of HDR Templates.

A 96-mer ssODN was designed spanning the target site of the ZFN and containing two base changes encoding the desired T448A conversion (FIG. 1) plus a third (silent) base change to assist in preventing ZFN re-cutting of any introgressed alleles. The plasmid DNA template was designed with the same three base changes as the ssODN at the ZFN target site, with additional single base changes encoding the S485P and S531P (FIG. 1). This 251 bp central domain containing all five base changes was flanked by homology arms of 626 bp and 799 bp, 5′ to the first base change and 3′ to the final base change respectively.

### Zygote Injection and Transfers.

Embryos were produced from Large-White gilts that were approximately 9 months of age and weighed at least 120 kg at time of use. Super-ovulation was achieved by feeding, between day 11 and 15 following an observed oestrus, 20 mg altrenogest (Regumate, Hoechst Roussel Vet Ltd) once daily for 4 days and 20 mg altrenogest twice on the 5th day. On the 6th day, 1500 IU of eCG (PMSG, Intervet UK Ltd) was injected at 20:00 hrs. Eighty-three hours later 750 IU hCG (Chorulon, Intervet UK Ltd) was injected. Donor gilts were inseminated twice 6 hours apart after exhibiting heat generated following super-ovulation. Embryos were surgically recovered from mated donors by mid-line laparotomy under general anaesthesia on day 1 following oestrus into NCSU-23 HEPES base medium. Embryos were subjected to a single 2-5 pl cytoplasmic injection of the pVAX single mRNAs at 2 ng/μL or 4 ng/μL with ssODN or plasmid template respectively. Recipient females were treated identically to donor gilts but remained un-mated. Following ZFN injection, fertilized embryos were transferred to recipient gilts following a mid-line laparotomy under general anaesthesia. During surgery, the reproductive tract was exposed and embryos were transferred into the oviduct of recipients using a 3.5 French gauge tomcat catheter. Litter sizes ranged from 1-13 piglets.

TABLE 2
Summary of pig zygote injections

| | No zygotes | | N^{o} live | | |
|---|---|---|---|---|---|
| Construct | injected | N^{o} recipients | piglets | NHEJ | HDR |
| ZFN + ssODN | 95 | 4 | 39 | 0 | 0 |
| ZFN + plasmid | 272 | 6 | 46 | 0 | 4 |

### Genotyping.

Genomic DNA was prepared from ear biopsy taken from piglets 2 days postpartum. PCR amplification with AccuPrime HiFi was conducted with primers oSL1 (gggtacaaagaggggtgagg—SEQ ID NO 13) which binds out-with the 5′ homology arm encoded by the plasmid and oSL2 (ctagctctgccctttccaga—SEQ ID NO 14) which binds within the 3′ homology arm of the plasmid. Cycling was 95° C. for 120 seconds then 40 cycles of 94° C. for 30 seconds, 59° C. for 30 seconds and 68° C. for 90 seconds, followed by primer extension of 68° C. for 5 minutes. Purified PCR products were directly sequenced.

### Reference Sequences

Sus scrofa v-rel avian reticuloendotheliosis viral oncogene homolog A (RELA), mRNA/cDNA sequence (NCBI Accession NM_001114281, Version NM_001114281.1), ZFN binding site is underlined and cut site is located between the two bases shown in bold.

| (SEQ ID NO 15) | |
|---|---|
| 1 | atggacgacc tcttccccct catcttcccc tcggagccgg ccccggcctc gggcccctat |
| 61 | gtggagatca tcgagcagcc caagcagcgg ggcatgcgct tccgctacaa gtgcgagggc |
| 121 | cgctcagccg gcagtatccc gggcgagagg agcacggata ccaccaagac ccaccccacc |
| 181 | atcaagatca atggctacac ggggccaggg acagtgcgca tctccctggt caccaaggac |
| 241 | ccccctcacc ggcctcaccc ccatgagctc gtggggaaag actgccggga tggcttctat |
| 301 | gaggctgagc tctgcccaga ccgctgcatc cacagcttcc agaacctggg gatccagtgt |
| 361 | gtaaagaagc gggacctgga acaggccatc aatcagcgca tccagaccaa caacaacccc |
| 421 | ttccaagttc ccatagaaga gcagcgcggg gactacgacc tgaatgctgt gcggctctgc |
| 481 | ttccaggtga cagtgcggga cccagcaggc aggcccctcc gcctgccgcc tgtcctctct |
| 541 | caccccatct ttgacaaccg tgcccccaac actgcagagc tcaagatctg ccgggtgaat |
| 601 | cggaactcgg ggagctgcct tgggggcgat gagatcttcc tgctgtgcga caaggtgcag |
| 661 | aaagaggaca tcgaggtgta tttcacgggc ccgggctggg aggcccgagg ctccttttca |
| 721 | caagccgacg tgcaccgaca agtggccatc gtgttccgga cgcctcccta cgcggacccc |
| 781 | agcctgcagg cccccgtgcg cgtctccatg cagctgcggc ggccttcgga tcgggagctc |
| 841 | agcgagccca tggaattcca gtacttgcca gacacagatg accggcaccg gattgaggag |
| 901 | aaacgcaaaa ggacctatga gacctttaag agcatcatga agaagagtcc tttcaatgga |
| 961 | cccaccgacc cccggcctgc aacccggcgc attgctgtgc cttcccgcag ctcagcttcc |
| 1021 | gtccccaagc cagctcccca gccctatccc tttacgccat ctctcagcac catcaacttt |
| 1081 | gacgagttca cgcccatggc ctttgcttct gggcagatcc caggccagac ctcagccttg |
| 1141 | gccccagccc ctgccccagt cctggtccag gccccagccc cggccccagc cccagccatg |
| 1201 | gcatcagctc tggcccaggc cccagcccct gtccccgtcc tagcccccgg ccttgctcag |
| 1261 | gctgtggccc cgcctgcccc taaaaccaac caggctgggg aagggacact gacagaggcc |
| 1321 | ctgctgcagc tgcagtttga tactgatgag gacctggggg ccctgctcgg caataacact |
| 1381 | gacccgaccg tgttcacgga cctggcatcc gtcgacaact ctgagtttca gcagctgctg |
| 1441 | aaccagggtg tatccatgcc cccccacaca gctgagccca tgctgatgga gtaccctgag |
| 1501 | gctataactc gcttggtgac agggtcccag agaccccctg acccagctcc cactcccctg |
| 1561 | ggggcctctg ggctcaccaa cggtctcctc tcgggggacg aagacttctc ctccattgcg |
| 1621 | gacatggact tctcagccct tctgagtcag atcagctcct aa |

Sus scrofa RELA, protein sequence (NCBI Reference Sequence: NP_001107753.1):
(SEQ ID NO 16)
MDDLFPLIFPSEPAPASGPYVEIIEQPKQRGMRFRYKCEGRSAGSIPGERSTDTTKTHPTIK
INGYTGPGTVRISLVTKDPPHRPHPHELVGKDCRDGFYEAELCPDRCIHSFQNLGIQCVKKR
DLEQAINQRIQTNNNPFQVPIEEQRGDYDLNAVRLCFQVTVRDPAGRPLRLPPVLSHPIFDN
RAPNTAELKICRVNRNSGSCLGGDEIFLLCDKVQKEDIEVYFTGPGWEARGSFSQADVHRQV
AIVFRTPPYADPSLQAPVRVSMQLRRPSDRELSEPMEFQYLPDTDDRHRIEEKRKRTYETFK
SIMKKSPFNGPTDPRPATRRIAVPSRSSASVPKPAPQPYPFTPSLSTINFDEFTPMAFASGQ
IPGQTSALAPAPAPVLVQAPAPAPAPAMASALAQAPAPVPVLAPGLAQAVAPPAPKTNQAGE
GTLTEALLQLQFDTDEDLGALLGNNTDPTVFTDLASVDNSEFQQLLNQGVSMPPHTAEPMLM
EYPEAITRLVTGSQRPPDPAPTPLGASGLTNGLLSGDEDFSSIADMDFSALLSQISS

### REFERENCES

1. Carroll, D. Genome engineering with targetable nucleases. Annual review of biochemistry 83, 409-439 (2014).
2. Urnov, F. D., Rebar, E. J., Holmes, M. C., Zhang, H. S. & Gregory, P. D. Genome editing with engineered zinc finger nucleases. Nature reviews. Genetics 11, 636-646 (2010).
3. Urnov, F. D. et al. Highly efficient endogenous human gene correction using designed zinc-finger nucleases. Nature 435, 646-651 (2005).
4. Fahrenkrug, S. C. et al. Precision genetics for complex objectives in animal agriculture. Journal of animal science 88, 2530-2539 (2010).
5. Konishi, S. et al. An SNP caused loss of seed shattering during rice domestication. Science (New York, N.Y.) 312, 1392-1396 (2006).
6. Palgrave, C. J. et al. Species-specific variation in RELA underlies differences in NFkappaB activity: a potential role in African swine fever pathogenesis. Journal of virology 85, 6008-6014 (2011).
7. Elliott, B., Richardson, C., Winderbaum, J., Nickoloff, J. A. & Jasin, M. Gene conversion tracts from double-strand break repair in mammalian cells. Molecular and cellular biology 18, 93-101 (1998).
8. Fang, J. et al. Stable antibody expression at therapeutic levels using the 2A peptide. Nature biotechnology 23, 584-590 (2005).
9. Perez, E. E. et al. Establishment of HIV-1 resistance in CD4+ T cells by genome editing using zinc-finger nucleases. Nature biotechnology 26, 808-816 (2008).
10. Guschin, D. Y. et al. A rapid and general assay for monitoring endogenous gene modification. Methods in molecular biology (Clifton, N.J.) 649, 247-256 (2010).
11. Hauschild, J. et al. Efficient generation of a biallelic knockout in pigs using zinc-finger nucleases. Proceedings of the National Academy of Sciences of the United States of America 108, 12013-12017 (2011).
12. Geurts, A. M. et al. Knockout rats via embryo microinjection of zinc-finger nucleases. Science (New York, N.Y.) 325, 433 (2009).
13. Carlson, D. F. et al. Efficient TALEN-mediated gene knockout in livestock. Proceedings of the National Academy of Sciences of the United States of America 109, 17382-17387 (2012).
14. Lillico, S. G. et al. Live pigs produced from genome edited zygotes. Scientific reports 3, 2847 (2013).
15. Proudfoot, C. et al. Genome edited sheep and cattle. Transgenic research 24, 147-153 (2015).
16. Chen, F. et al. High-frequency genome editing using ssDNA oligonucleotides with zinc-finger nucleases. Nature methods 8, 753-755 (2011).
17. Cui, C. et al. Gene targeting by TALEN-induced homologous recombination in goats directs production of beta-lactoglobulin-free, high-human lactoferrin milk. Scientific reports 5, 10482 (2015).
18. Ni, W. et al. Efficient gene knockout in goats using CRISPR/Cas9 system. PloS one 9, e106718 (2014).
19. Hai, T., Teng, F., Guo, R., Li, W. & Zhou, Q. One-step generation of knockout pigs by zygote injection of CRISPR/Cas system. Cell research 24, 372-375 (2014).

1. A genetically-edited swine comprising an introgressed heterologous nucleic acid sequence in the RELA gene.

2. The genetically-edited swine of claim 1 wherein the swine is a domestic pig.

3. The genetically-edited swine of claim 1 wherein the introgressed heterologous nucleic acid sequence comprises a heterologous RELA allele.

4. The genetically-edited swine of claim 3 wherein the introgressed heterologous sequence comprises a trans-species heterologous RELA allele.

5. The genetically-edited swine of claim 1 wherein the introgressed heterologous nucleic acid sequence converts a wild-type RELA allele to the corresponding warthog RELA allele.

6. The genetically-edited swine of claim 1 which comprises an introgressed RELA allele that differs from the wild-type RELA allele sequence by changes in two or more bases.

7. The genetically-edited swine of claim 1 which comprises an introgressed heterologous nucleic acid which is 50 or more bases in length.

8. The genetically-edited swine of claim 1 wherein all cells of the genetically-edited swine contain the introgressed heterologous nucleic acid sequence.

9. The genetically-edited swine of claim 1 wherein the introgressed heterologous nucleic acid sequence changes the sequence of the RELA protein.

10. The genetically-edited swine of claim 1 wherein the introgressed heterologous nucleic acid sequence changes sequences in the region of the RELA gene which encodes the transactivation domain of RELA.

11. The genetically-edited swine of claim 10 wherein the introgressed heterologous nucleic acid sequence changes the region of the RELA gene which encodes amino acids 448 to 531 of RELA.

12. The genetically-edited swine of claim 1 which comprises an introgressed trans-species allele of the RELA gene.

13. The genetically-edited swine of claim 1 which is a domestic pig comprising an introgressed warthog RELA gene.

14. The genetically-edited swine of claim 1 wherein the swine is bi-allelic for an introgressed RELA allele.

15. The genetically-edited swine of claim 1 which is a domestic pig and wherein the introgressed heterologous nucleic acid sequence causes a change in one or more of the following amino acids of RELA:
T448;
S485; and
S531.

16. The genetically-edited swine of claim 15 wherein all three amino acids are changed.

17. The genetically-edited swine of claim 15 comprising at least one of the following amino acid changes to RELA: T448A, S485P, and S531P.

18. The genetically-edited swine of claim 1 which is a domestic pig that comprises an introgressed heterologous nucleic acid sequence which results in the following amino acid changes to RELA: T448A, S485P and S531P.

19. The genetically-edited swine of claim 18 in which no amino acid changes other than T448A, S485P and S531P of RELA are caused by the introgressed heterologous nucleic acid.

20. The genetically-edited swine of claim 1 which is a domestic pig wherein the RELA gene has been edited such that it encodes the sequence as set out below:
(SEQ ID NO 17)
LLQLQFDADEDLGALLGNNTDPTVFTDLASVDNSEFQQLLNQGVPMPPHT
AEPMLMEYPEAITRLVTGSQRPPDPAPTPLGASGLTNGLLPDGEDFSSIA
DM.

21. The genetically-edited swine of claim 1 which is a domestic pig that has improved tolerance to ASFV infection resulting from the introgressed heterologous nucleic acid sequence.

22. A cell nucleus, germ cell, stem cell, gamete, blastocyst, embryo, foetus and/or donor cell of a swine comprising an introgressed heterologous nucleic acid sequence in the RELA gene.

23. A cell nucleus, germ cell, stem cell, gamete, blastocyst, embryo, foetus and/or donor cell of a swine according to claim 22 which comprises an introgressed warthog RELA allele.

24. A method of producing a genetically-edited swine having an introgressed heterologous nucleic acid sequence in the RELA gene, the method comprising the steps of:
providing a swine zygote;
introducing into said zygote a site-specific nuclease, the nuclease being adapted to target a desired genomic sequence in the RELA gene to be edited, and to introduce a double stranded break;
introducing a template nucleic acid comprising the heterologous nucleic acid sequence to be introgressed into the RELA gene, the heterologous sequence being flanked by sequences homologous to genomic RELA sequences;
incubating said zygote under suitable conditions to permit cutting of the genome by the site-specific nuclease and introgression of the heterologous nucleic acid sequence into the RELA gene by homology directed repair; and
generating an animal from said zygote.

25. The method of claim 24 wherein the swine is a domestic pig.

26. The method of claim 24 wherein the site-specific nuclease is adapted to target and cut within the region of the RELA gene encoding the transactivation domain of RELA.

27. The method of claim 24 wherein the site-specific nuclease is adapted to target and cut within exon 9 of the RELA gene.

28. The method of claim 27 wherein the site-specific nuclease is adapted to target and cut upstream of the region of the RELA gene encoding amino acid T448.

29. The method of claim 24 wherein the site-specific nuclease is adapted to target and cut a sequence of the RELA gene lying between bases 1200 and 1341 with reference to SEQ ID NO 15.

30. The method of claim 24 wherein the site-specific nuclease comprises a pair of cooperating site-specific nucleases and the target site of one of the pair is GATACTGATGAGGAC (SEQ ID NO 18) and the target site of the other of the pair is CTCCGGGACGACGTC (SEQ ID NO 19).

31. The method of claim 24 wherein mRNA encoding the nuclease is introduced into the zygote.

32. The method of claim 24 wherein introgression of the heterologous nucleic acid sequence is completed in the zygote at the single cell stage.

33. The method of claim 24 in which the template nucleic acid comprises a region including the heterologous nucleic acid sequence flanked on each side by homologous sequences.

34. The method of claim 24 in which the template construct comprises a heterologous nucleic acid that is 50 or more bases in length.

35. The method of claim 24 in which the template nucleic acid comprises two or more base changes compared with the corresponding genomic nucleic acid sequence at the target site for the SSN site-specific nuclease.

36. The method of claim 24 in which the template nucleic acid comprises a region including the warthog RELA haplotype flanked on each side by homologous sequences.

37. The method of claim 24 which comprises introgressing a heterologous nucleic acid sequence that results in the following amino acid changes to RELA: T448A, S485P and S531P.

38. The method of claim 24 in which the template nucleic acid comprises a region including the warthog RELA of 251 or more nucleotides in length, which comprises a nucleic acid sequence encoding the protein sequence:
(SEQ ID NO 23)
ADEDLGALLGNNTDPTVFTDLASVDNSEFQQLLNQGVPMPPHTAEPMLMEY
PEAITRLVTGSQRPPDPAPTPLGASGLTNGLLP flanked by
homologous regions.

39. The method of claim 24 in which the template nucleic acid is double stranded.

40. The method of claim 24 in which the template nucleic acid is provided in a plasmid.

41. The method of claim 24 in which the template is plasmid comprising a 251 bp region containing the warthog RELA haplotype flanked by homology arms of 626 bp and 799 bp.

42. The method of claim 24 comprising the steps of:
providing a domestic pig zygote;
introducing into said zygote a pair of cooperating site-specific nucleases, the nucleases being adapted to target the RELA gene in the region of within 20 bp of the sequence encoding T448A and introduce a double stranded break;
introducing a template nucleic acid comprising a heterologous nucleic acid comprising a sequence encoding the corresponding warthog RELA haplotype flanked by sequences homologous to the genomic RELA sequence of the pig;
incubating said zygote under suitable conditions to permit cutting of the genome by the site-specific nuclease and introgression of the heterologous nucleic acid by homology-directed repair; and
generating a pig from said zygote.


SEQUENCE LISTING (23 sequences)

SEQ ID NO: 1 - DNA, 41 bp, Sus scrofa
agaggccctgctgcagctgcagtttgatactgatgaggacc

SEQ ID NO: 2 - DNA, 41 bp, Sus scrofa
tctccgggacgacgtcgacgtcaaactatgactactcctgg

SEQ ID NO: 3 - protein, 7 aa, Artificial Sequence
  ZFN DNA recognition helicase
DRSDLSR

SEQ ID NO: 4 - protein, 7 aa, Artificial Sequence
  ZFN DNA recognition helicase
RSDNLTR

SEQ ID NO: 5 - protein, 7 aa, Artificial Sequence
  ZFN DNA recognition helicase
TSGNLTR

SEQ ID NO: 6 - protein, 7 aa, Artificial Sequence
  ZFN DNA recognition helicase
LRQDLNK

SEQ ID NO: 7 - protein, 7 aa, Artificial Sequence
  ZFN DNA recognition helicase
TSSNLSR

SEQ ID NO: 8 - protein, 7 aa, Artificial Sequence
  ZFN DNA recognition helicase
AMQTLRV

SEQ ID NO: 9 - protein, 7 aa, Artificial Sequence
  ZFN DNA recognition helicase
DRSHLAR

SEQ ID NO: 10 - protein, 7 aa, Artificial Sequence
  ZFN DNA recognition helicase
RSDNLSE

SEQ ID NO: 11 - protein, 7 aa, Artificial Sequence
  ZFN DNA recognition helicase
KRCNLRC

SEQ ID NO: 12 - protein, 7 aa, Artificial Sequence
  ZFN DNA recognition helicase
RSAVLSE

SEQ ID NO: 13 - DNA, 20 bp, Artificial Sequence
  PCR primer oSL1 sequence
gggtacaaagaggggtgagg

SEQ ID NO: 14 - DNA, 20 bp, Artificial Sequence
  PCR primer oSL2 sequence
ctagctctgccctttccaga

SEQ ID NO: 15 - DNA, 1662 bp, Sus scrofa
atggacgacctcttccccctcatcttcccctcggagccggccccggcctcgggcccctatgtggagatcatcgagcagcccaagcagcggggcatgcgcttccgctacaagtgcgagggccgctcagccggcagtatcccgggcgagaggagcacggataccaccaagacccaccccaccatcaagatcaatggctacacggggccagggacagtgcgcatctccctggtcaccaaggacccccctcaccggcctcacccccatgagctcgtggggaaagactgccgggatggcttctatgaggctgagctctgcccagaccgctgcatccacagcttccagaacctggggatccagtgtgtaaagaagcgggacctggaacaggccatcaatcagcgcatccagaccaacaacaaccccttccaagttcccatagaagagcagcgcggggactacgacctgaatgctgtgcggctctgcttccaggtgacagtgcgggacccagcaggcaggcccctccgcctgccgcctgtcctctctcaccccatctttgacaaccgtgcccccaacactgcagagctcaagatctgccgggtgaatcggaactcggggagctgccttgggggcgatgagatcttcctgctgtgcgacaaggtgcagaaagaggacatcgaggtgtatttcacgggcccgggctgggaggcccgaggctccttttcacaagccgacgtgcaccgacaagtggccatcgtgttccggacgcctccctacgcggaccccagcctgcaggcccccgtgcgcgtctccatgcagctgcggcggccttcggatcgggagctcagcgagcccatggaattccagtacttgccagacacagatgaccggcaccggattgaggagaaacgcaaaaggacctatgagacctttaagagcatcatgaagaagagtcctttcaatggacccaccgacccccggcctgcaacccggcgcattgctgtgccttcccgcagctcagcttccgtccccaagccagctccccagccctatccctttacgccatctctcagcaccatcaactttgacgagttcacgcccatggcctttgcttctgggcagatcccaggccagacctcagccttggccccagcccctgccccagtcctggtccaggccccagccccggccccagccccagccatggcatcagctctggcccaggccccagcccctgtccccgtcctagcccccggccttgctcaggctgtggccccgcctgcccctaaaaccaaccaggctggggaagggacactgacagaggccctgctgcagctgcagtttgatactgatgaggacctgggggccctgctcggcaataacactgacccgaccgtgttcacggacctggcatccgtcgacaactctgagtttcagcagctgctgaaccagggtgtatccatgcccccccacacagctgagcccatgctgatggagtaccctgaggctataactcgcttggtgacagggtcccagagaccccctgacccagctcccactcccctgggggcctctgggctcaccaacggtctcctctcgggggacgaagacttctcctccattgcggacatggacttctcagcccttctgagtcagatcagctcctaa

SEQ ID NO: 16 - protein, 553 aa, Sus scrofa
MDDLFPLIFPSEPAPASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAINQRIQTNNNPFQVPIEEQRGDYDLNAVRLCFQVTVRDPAGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFNGPTDPRPATRRIAVPSRSSASVPKPAPQPYPFTPSLSTINFDEFTPMAFASGQIPGQTSALAPAPAPVLVQAPAPAPAPAMASALAQAPAPVPVLAPGLAQAVAPPAPKTNQAGEGTLTEALLQLQFDTDEDLGALLGNNTDPTVFTDLASVDNSEFQQLLNQGVSMPPHTAEPMLMEYPEAITRLVTGSQRPPDPAPTPLGASGLTNGLLSGDEDFSSIADMDFSALLSQISS

SEQ ID NO: 17 - protein, 102 aa, Artificial Sequence
  Protein encoded by the edited RELA gene
LLQLQFDADEDLGALLGNNTDPTVFTDLASVDNSEFQQLLNQGVPMPPHTAEPMLMEYPEAITRLVTGSQRPPDPAPTPLGASGLTNGLLPDGEDFSSIADM

SEQ ID NO: 18 - DNA, 15 bp, Sus scrofa
gatactgatgaggac

SEQ ID NO: 19 - DNA, 15 bp, Sus scrofa
ctccgggacgacgtc

SEQ ID NO: 20 - DNA, 15 bp, Artificial Sequence
  nucleic acid sequence which prevents recutting by the SSN
gatgcagacgaggac

SEQ ID NO: 21 - DNA, 23 bp, Sus scrofa
tgcagtttgatactgatgaggac

SEQ ID NO: 22 - DNA, 23 bp, Artificial Sequence
  post-introgression sequence
tgcagtttgatgcagacgaggac

SEQ ID NO: 23 - protein, 84 aa, Artificial Sequence
  Protein encoded by the edited RELA gene
ADEDLGALLGNNTDPTVFTDLASVDNSEFQQLLNQGVPMPPHTAEPMLMEYPEAITRLVTGSQRPPDPAPTPLGASGLTNGLLP